(19) United States
(12) Patent Application Publication
(10) Pub. No.: US 2018/0238867 A1
(43) Pub. Date: Aug. 23, 2018

(54) DETECTION DEVICE AND TARGET DETECTION METHOD USING THE SAME

(71) Applicant: NEC Solution Innovators, Ltd., Tokyo (JP)

(72) Inventors: Katsunori HORII, Tokyo (JP); Naoto KANEKO, Tokyo (JP)

(73) Assignee: NEC Solution Innovators, Ltd., Tokyo (JP)

(21) Appl. No.: 15/771,962

(22) PCT Filed: Oct. 27, 2016
(86) PCT No.: PCT/JP2016/081879

§ 371 Date: Apr. 27, 2018

(30) Foreign Application Priority Data
Oct. 30, 2015 (JP) 2015-214649

(51) Int. Cl. G01N 33/543; G01N 27/414

(52) U.S. Cl. CPC G01N 33/54306; G01N 27/4145; G01N 33/54373; G01N 33/54386

(57) ABSTRACT

The present invention provides a new detection device and a target detection method using the same.
The detection device of the present invention includes a transistor provided with a nucleic acid sensor. The nucleic acid sensor includes a conformation-forming region (D) that forms a predetermined conformation and a binding region (A) that binds to a target. In the absence of the target, the conformation-forming region (D) is inhibited from forming the conformation. In the presence of the target, upon contact of the target to the binding region (A), the conformation-forming region (D) forms the conformation. In a state where the conformation is formed, the number of nucleotide residues that compose the nucleic acid sensor within a range of Debye length of the transistor increases or decreases as compared to a state where formation of the conformation is inhibited.

### TECHNICAL FIELD

The present invention relates to a detection device and a target detection method using the same.

### BACKGROUND ART

In various fields such as fields of clinical medical care, food, and environment, there is a demand for detecting a target. For the detection of a target, methods of utilizing interaction with the target are commonly used.

As a method of utilizing the interaction, known is a method of detecting the target by detecting a change in a charge of the target caused upon binding between the binding substance and the target using a transistor in which a binding substance that binds to the target is disposed (Non-Patent Document 1).

### CITATION LIST

### Non-Patent Document(s)

Non-Patent Document 1: Sho Hideshima, et. al., “Attomolar Detection of Influenza A Virus Hemagglutinin Human H1 and Avian H5 Using Glycan-Blotted Field Effect Transistor Biosensor”, 2013, Analytical Chemistry, vol.85, pp. 5641 to 5644

### DISCLOSURE OF THE INVENTION

### Problem to be Solved by the Invention

The method using the transistor, however, has a problem that a target having a charge can be analyzed but a target having no or almost no charge cannot be analyzed.

Hence, the present invention is intended to provide a new detection device and a target detection method using the same.

### Means for Solving Problem

The present invention provides a detection device including a transistor provided with a nucleic acid sensor, wherein the nucleic acid sensor includes a conformation-forming region (D) that forms a predetermined conformation and a binding region (A) that binds to a target, in the absence of the target, the conformation-forming region (D) is inhibited from forming the conformation, in the presence of the target, upon contact of the target to the binding region (A), the conformation-forming region (D) forms the conformation, and in a state where the conformation is formed, the number of nucleotide residues that compose the nucleic acid sensor within a range of Debye length of the transistor increases or decreases as compared to a state where formation of the conformation is inhibited.

The present invention provides a method for detecting a target including the steps of: bringing a sample into contact with the detection device of the present invention; and detecting increase or decrease of the number of nucleotide residues that compose the nucleic acid sensor within the range of Debye length of the detection device to detect a target in the sample.

### Effects of the Invention

According to the detection device and target detection method using the same of the present invention, a target can be detected.

### BRIEF DESCRIPTION OF DRAWINGS

FIG. 1 is a schematic view showing the structural change of the nucleic acid sensor in the device of the present invention.

FIG. 2 is a schematic view showing the structural change of the nucleic acid sensor in the device of the present invention.

### DESCRIPTION OF EMBODIMENTS

<Detection device>

The detection device of the present invention (hereinafter, also referred to as a “device”) is, as described above, characterized in that it includes a transistor provided with a nucleic acid sensor (hereinafter, also referred to as a “ sensor”), wherein the nucleic acid sensor includes a conformation-forming region (D) that forms a predetermined conformation (hereinafter, also referred to as a “predetermined structure”) and a binding region (A) that binds to a target, in the absence of the target, the conformation-forming region (D) is inhibited from forming the conformation, in the presence of the target, upon contact of the target to the binding region (A), the conformation-forming region (D) forms the conformation, and in a state where the conformation is formed, the number of nucleotide residues that compose the nucleic acid sensor within a range of Debye length of the transistor increases or decreases as compared to a state where formation of the conformation is inhibited.

In the sensor disposed in the transistor, the number of nucleotide residues that compose the sensor increases or decreases within the range of Debye length (hereinafter, also referred to as “the number of nucleotides within Debye length”) in the presence of the target (i.e., in a state where the predetermined structure is formed) as described below. The nucleotide residues that compose the sensor have, for example, a negative charge. Thus, in the presence of the target, for example, the charge within the range of Debye length decreases or increases so as to correspond to increase or decrease of the number of nucleotides within Debye length as compared to in the absence of the target. That is, in the detection device of the present invention, for example, the charge within the range of Debye length increases or decreases due to the presence of the target irrespective of the charge of the target. Therefore, a target having no or almost no charge can be analyzed. Note that, since the nucleotide residues that compose the sensor include bases, sugar skeletons, and phosphate groups, the number of nucleotide residues can be also referred to as, for example, “the number of bases”, “the number of sugar skeletons”, and “the number of phosphate groups”.

Hereinafter, each region is also referred to as a nucleic acid region. In the present invention, the single stranded nucleic acid sensor described below can be also referred to as, for example, a single stranded sensor and the double stranded nucleic acid sensor described below can be also referred to as, for example, a double stranded sensor. The phenomenon in which the conformation-forming region (D) is inhibited from forming a predetermined structure is also referred to as “switch-OFF” (or “turn-OFF”) and phenomenon in which the conformation-forming region (D) forms the predetermined structure is also referred to as “switch-ON” (or “turn-ON”).

The conformation-forming region (D) is a nucleic acid region that forms a predetermined structure. The predetermined structure is not limited to particular structures, and can be, for example, a higher order structure composed of nucleic acid molecules. Specific examples thereof include a secondary structure, a tertiary structure, and a quaternary structure. Specific examples of the predetermined structure include a stem structure, a hairpin loop structure, a bulge loop structure, a G-quartet structure, an i-motif structure, and a pseudoknot structure. Specifically, the conformation-forming region (D) is, for example, a G-forming region (G) that forms a G-quartet structure, and the predetermined structure is a G-quartet structure. In the conformation-forming region (D), the number of predetermined structures to be formed is not particularly limited, and is, for example, 1 to 10. It is only required that the sequence of the conformation-forming region (D) forms the predetermined structure. The conformation-forming region (D) may form a conformation (hereinafter, also referred to as “other conformation”) other than the predetermined structure in the absence of the target, for example. In this case, for example, in the nucleic acid sensor, the conformation-forming region (D) may form other conformation in the absence of the target and the conformation-forming region (D) may form the predetermined conformation upon contact of the target to the binding region (A) in the presence of the target. The “other conformation” is, for example, a conformation that is different from the predetermined structure. Regarding the specific examples of the “other conformation”, for example, reference can be made to the specific examples of the predetermined structure.

The G-quartet (also referred to as a “G-tetrad”) is commonly known as a G (guanine) tetrameric planar structure. The G-forming region (G) includes a plurality of G bases and forms a G-quartet structure composed of plurality of G bases in its region, for example. The G-quartet structure may be, for example, either a parallel type or an antiparallel type, and is preferably a parallel type. In the sensor of the present invention, the number of G-quartet structures to be formed in the G-forming region (G) is not particularly limited, and can be, for example, 1 or 2 or more. Preferably, the G-forming region (G) forms a guanine quadruplex structure in which two or more G-quartets stack on top of each other. The sequence of the G-forming region (G) is not limited to particular sequences as long as it forms the G-quartet structure and is preferably a sequence that forms a guanine quadruplex structure.

As the sequence of the G-forming region (G), for example, the sequence of a publicly known nucleic acid molecule that forms the G-quartet structure can be used. The publicly known nucleic acid molecule can be, for example, the nucleic acid molecules described in the following research paper (1) to (4).
(1) Travascio et al., Chem. Biol., 1998, vol.5, pp. 505 to 517
(2) Cheng et al., Biochemistry, 2009, vol.48, pp. 7817 to 7823
(3) Teller et al., Anal. Chem., 2009, vol.81, pp. 9114 to 9119
(4) Tao et al., Anal. Chem., 2009, vol.81, pp. 2144 to 2149

When the predetermined conformation is an i-motif structure, for example, the sequence of a publicly known nucleic acid molecule that forms the i-motif structure can be used as the sequence of the conformation-forming region (D). The publicly known nucleic acid molecule can be, for example, the nucleic acid molecule described in the following research paper (5).
(5) Patrycja Bielecka et al., “Fluorescent Sensor for PH Monitoring Based on an i-Motif-Switching Aptamer Containing a Tricyclic Cytosine Analogue (tC)”, 2015, Molecules, vol.20, pp.18511 to 18525

When the predetermined conformation is a pseudoknot structure, for example, the sequence of a publicly known nucleic acid molecule that forms the pseudoknot structure can be used as the sequence of the conformation-forming region (D). The publicly known nucleic acid molecule can be, for example, the nucleic acid molecule described in the following research paper (6).
(6) Calliste Reiling et al., “Loop Contributions to the Folding Thermodynamics of DNA Straight Hairpin Loops and Pseudoknots”, 2015, J. Phys. Chem. B, vol.119, pp.I 939 to 1946

The conformation-forming region (D) may be, for example, either a single stranded type or a double stranded type. The single stranded type can form a predetermined structure in a single stranded conformation-forming region (D), for example. The double stranded type includes a first region (D1) and second region (D2) and can form a predetermined structure between the first region (D1) and the second region (D2), for example. The latter double stranded type can be, for example, a structure in which the first region and the second region are indirectly linked, and the details of which are described in the nucleic acid sensor (iv) described below.

The length of the single stranded conformation-forming region (D) is not particularly limited, and the lower limit thereof is, for example, 11-mer, 13-mer, or 15-mer and the upper limit thereof is, for example, 60-mer, 36-mer, or 18-mer.

In the double stranded conformation-forming region (D), the lengths of the first region (D1) and second region (D2) are not particularly limited and may be identical to or different from each other. The length of the first region (D1) is not particularly limited, and the lower limit thereof is, for example, 7-mer, 8-mer, or 10-mer, the upper limit thereof is, for example, 30-mer, 20-mer, or 10-mer, and the length is, for example, in the range from 7 to 30-mer, 7 to 20-mer, or 7 to 10-mer. The length of the second region (D2) is not particularly limited, and the lower limit thereof is, for example, 7-mer, 8-mer, or 10-mer, the upper limit thereof is, for example, 30-mer, 20-mer, or 10-mer, and the length is, for example, in the range from 7 to 30-mer, from 7 to 20-mer, or from 7 to 10-mer.

In the present invention, a target is not limited to particular targets and any target can be selected. In accordance with a selected target, a binding nucleic acid molecule that binds to the target can be used as the binding region (A).

The target is not particularly limited, and examples thereof include low-molecular compounds, microorganisms, viruses, food allergen, agricultural chemicals, mycotoxin, and antibodies. Examples of the low-molecular compound include melamine, antibiotics, agricultural chemicals, and endocrine-disrupting chemicals. Examples of the microorganism include Salmonella enterica, Listeria monocytogenes, Escherichia coli, and mold. The virus is, for example, norovirus.

The length of the binding region (A) is not particularly limited, and the lower limit thereof is, for example, 12-mer, 15-mer, or 18-mer, the upper limit thereof is, for example, 140-mer, 80-mer, or 60-mer, and the length is, for example, in the range from 12 to 140-mer, from 15 to 80-mer, or from 18 to 60-mer.

In the present invention, the state where a sequence is complementary to another sequence means, for example, the state where annealing is allowed between these sequences. The annealing is also called stem formation, for example. In the present invention, for example, the state where a sequence is complementary to another sequence is the state where the complementarity in alignment of two kinds of sequences is, for example, at least 90%, at least 95%, at least 96%, at least 97%, at least 98%, or at least 99% and is preferably 100% (i.e., complete complementary state). Also, in the nucleic acid sensor, the state where a sequence is complementary to another sequence means, for example, that the bases of one of the sequences from the 5′ side to the 3′ side are complementary to the bases of the other of the sequences from the 3′ side to the 5′ side.

In the present invention, the sensor can be, for example, the following sensors (I) and (II). In the present invention, one kind or two or more kinds of the sensors may be disposed in the transistor, for example.

The sensors (I) and (II) are described below as examples of the sensor. In the following sensors, the predetermined conformation is preferably a G-quartet structure, for example. Note that, regarding the following sensors, reference can be made to the description as to each sensor, unless otherwise noted. In the description of the sensors (I) and (II) below, a “conformation” denotes a “predetermined conformation”.

1. Nucleic Acid Sensor (I)

The nucleic acid sensor (I) is, for example, a double stranded nucleic acid sensor composed of a first strand (ss1) and a second strand (ss2). The first strand (ss1) includes the conformation-forming region (D) and the binding region (A) in this order. The second strand (ss2) includes a stem-forming region (S_{D}) and a stem-forming region (S_{A}) in this order. The stem-forming region (S_{D}) includes a sequence complementary to the conformation-forming region (D). The stem-forming region (S_{A}) includes a sequence complementary to the binding region (A). In the absence of the target, the conformation-forming region (D) is inhibited from forming the conformation and hybridizes to the second strand (ss2). In the presence of the target, upon contact of the target to the binding region (A) of the first strand (ss1), the conformation-forming region (D) forms the conformation and the first strand (ss1) is dissociated from the second strand (ss2). In the state where the conformation is formed, the number of nucleotide residues that compose the nucleic acid sensor within a range of Debye length of the transistor decreases as compared to the state where formation of the conformation is inhibited.

In the nucleic acid sensor (I), the conformation-forming region (D) is, for example, the single stranded type.

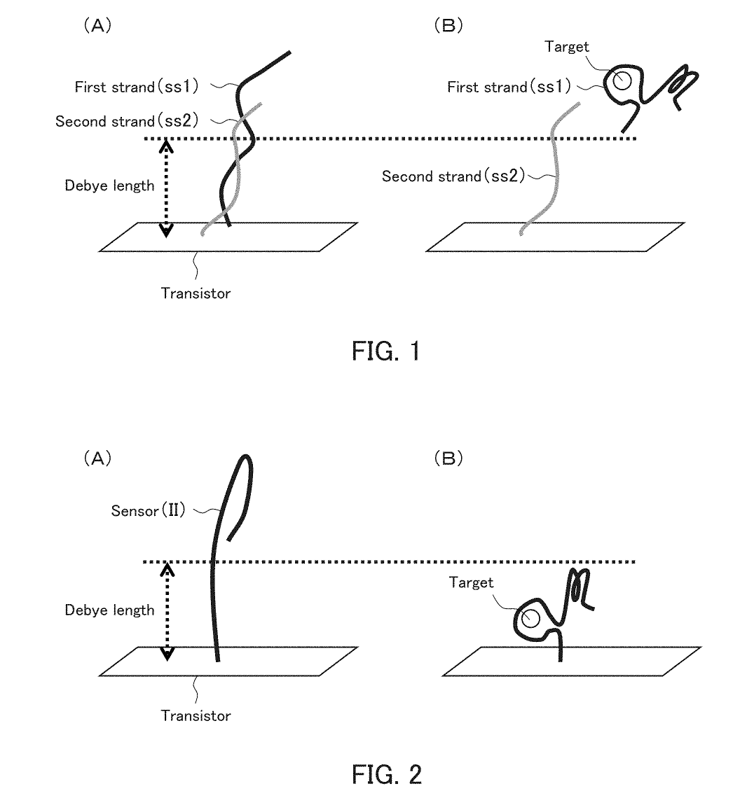

As shown in FIG. 1, in the sensor (I), based on the mechanism described below, it is presumed that formation of the conformation in the conformation-forming region (D) is controlled to be ON or OFF depending on the presence or absence of a target, thereby decreasing the number of nucleotide residues that compose the sensor within the range of Debye length of the transistor, for example. The present invention, however, is not limited to this mechanism. It is commonly considered that a nucleic acid sequence thermodynamically fluctuates between possible structures to be formed, and the abundance ratio of a structure having relatively high stability is high. It is commonly known that, in the presence of a target, upon contact with the target, a binding nucleic acid molecule (binding region) such as an aptamer changes into a more stable structure and binds to the target. As to the conformation of a nucleic acid sequence such as a G-quartet structure, it is also commonly considered that the abundance ratio of a structure having relatively high stability is high. As shown in (A) of FIG. 1, in the sensor (I), in the absence of a target, in response to annealing between the conformation-forming region (D) of the first strand (ss1) and the stem-forming region (SD) of the second strand (ss2), the conformation-forming region (D) is inhibited from forming the conformation (switch-OFF). Furthermore, in response to annealing between the binding region (A) of the first strand (ss1) and the stem-forming region (S_{A}) of the second strand (ss2), the binding region (A) is blocked from forming a more stable structure for binding to a target and keeps a structure of not binding to a target. On the other hand, in the sensor (I), in the presence of a target, upon contact of the target to the binding region (A), the annealing between the binding region (A) and the stem-forming region (S_{A}) is released, thereby changing the binding region (A) into a more stable structure. In accordance with this, the annealing between the conformation-forming region (D) and the stem-forming region (S_{D}) is released, thereby forming the conformation in the conformation-forming region (D) (switch-ON). As shown in (B) of FIG. I, in response to release of the annealing between the binding region (A) and the stem-forming region (SA) and release of the annealing between the conformation-forming region (D) and the stem-forming region (S_{D}), the first strand (ss1) is dissociated from the second strand (ss2). As a result, the first strand (ss1) can move out of the range of Debye length of the transistor. Thus, according to the sensor (I), since the number of nucleotides within Debye length in the presence of the target (i.e., in the state where the conformation is formed) decreases as compared to in the absence of the target (i.e., in the state where formation of the conformation is inhibited), a target analysis such as a qualitative analysis or a quantitative analysis can be achieved. Note that, although FIG. 1 shows the transistor in which the second strand (ss2) is disposed as an example, the first strand (ss1) may be disposed in the transistor as described below.

As described above, the sensor (I) includes the first strand (ss1) and the second strand (ss2). In the presence of the target, the first strand (ss1) or the second strand (ss2) are dissociated and the first strand (ss1) or the second strand (ss2) moves out of the range of Debye length of the transistor, for example. Thus, also in the case where the target has a charge, the charge within the range of Debye length varies according to the number of nucleic molecules of the dissociated first strand (ss1) or second strand (ss2) in the presence of the target. Thus, the sensor (I) is less affected by the charge of the target and is superior in general versatility, for example.

Preferably, the whole or a part of the stem-forming region (S_{D}) has a sequence complementary to a part of the conformation-forming region (D), for example. Furthermore, preferably, the whole or a part of the stem-forming region (S_{A}) has a sequence complementary to a part of the binding region (A), for example.

In the sensor (I), the order of the regions can be any order as long as annealing between the conformation-forming region (D) and the stem-forming region (S_{D}) and annealing between the binding region (A) and the stem-forming region (S_{A}) are allowed. Specific examples of the order are as follows.
(1) ss1 5′-A-D-3′ss2 3′-SA-SD-5′
(2) ss1 5′-D-A-3′ss2 3v-So-SA-5′

In the embodiment (1), preferably, the stem-forming region (S_{A}) is complementary to the 3′ side region of the binding region (A) and the stem-forming region (S_{D}) is complementary to the 5′ side region of the conformation-forming region (D). In the embodiment (2), preferably, the stem-forming region (S_{D}) is complementary to the 3′ side region of the conformation-forming region (D) and the stem-forming region (S_{A}) is complementary to the 5′ side region of the binding region (A).

In the sensor (I), for example, the regions may be linked to each other directly or indirectly. The direct linkage denotes the state in which the 3′ end of one of the regions and the 5′ end of the other of the regions are linked directly, for example, and the indirect linkage denotes the state in which the 3′ end of one of the regions and the 5′ end of the other of the regions are linked indirectly through an intervening linker region, for example. The intervening linker region may be, for example, a nucleic acid sequence or a non-nucleic acid sequence and is preferably the former.

Preferably, the sensor (I) includes the intervening linker region between the binding region (A) and the conformation-forming region (D) in the first strand (ss1) and includes the intervening linker region between the stem-forming region (S_{D}) and the stem-forming region (S_{A}) in the second strand (ss2), for example. Preferably, the intervening linker region (L₁) in the first strand (ss1) has a sequence noncomplementary to the intervening linker region (L₂) in the second strand (ss2).

Specific examples in which each of the embodiments (1) and (2) includes the intervening linker region in each of the first strand (ss1) and the second strand (ss2) are as follows. In the following examples, the intervening linker region that links the binding region (A) and the conformation-forming region (D) is referred to as (L₁) and the intervening linker region that links the stem-forming region (S_{D}) and the stem-forming region (S_{A}) is referred to as (L₂). The sensor (I) may include both of the (L₁) and the (L₂) or either one of them as intervening linker region(s), for example.
(1′) ss1 5′-A-L₁-D-3′ss2 3′-S_{A}-L₂-SD-5′
(2′) ss1 5′-D-L₁-A-3′ss2 3′- S_{D}-L2-SA -5′

In the embodiments (1′) and (2′), for example, ON-OFF of formation of a conformation is controlled as described below. In the absence of a target, for example, the binding region (A) and the stem-forming region (S_{A}) form a stem, the conformation-forming region (D) and the stem-forming region (S_{D}) form a stem, and the intervening linker region (L₁) and the intervening linker region (L₂) form an internal loop between these stems, thereby inhibiting the conformation-forming region (D) from forming a conformation. In the presence of a target, upon contact of the target to the binding region (A), formation of each stem is released and the conformation is formed in the conformation-forming region (D).

In the sensor (I), the length of each of the stem-forming region (S_{A}) and the stem-forming region (S_{D}) is not particularly limited. The length of the stem-forming region (S_{A}) is, for example, 1 to 60-mer, 1 to 10-mer, or 1 to 7-mer. The length of the stem-forming region (S_{D}) is, for example, 1 to 30-mer, 0 to 10-mer, 1 to 10-mer, 0 to 7-mer, or 1 to 7-mer. The length of the stem-forming region (S_{A}) and the length of the stem-forming region (S_{D}) may be the same, the former may be longer than the latter, or the latter may be longer than the former.

The length of each of the intervening linker regions (L₁) and (L₂) is not particularly limited. The length of each of the intervening linker regions (L₁) and (L₂) is, for example, 0 to 30-mer, 1 to 30-mer, 1 to 15-mer, or 1 to 6-mer. The length of the intervening linker region (L₁) and the length of the intervening linker region (L₂) may be identical to or different from each other, for example. In the latter case, the difference between the length of the intervening linker region (L₁) and the length of the intervening linker region (L₂) is not particularly limited and is, for example, 1 to 10-mer, 1 or 2-mer, or 1-mer.

In the sensor (I), the length of each of the first strand (ss1) and the second strand (ss2) is not particularly limited. The length of the first strand (ss1) is, for example, 40 to 200-mer, 42 to 100-mer, or 45 to 60-mer. The length of the second strand (ss2) is, for example, 4 to 120-mer, 5 to 25-mer, or 10 to 15-mer.

In the sensor (I), for example, the first strand (ss1) and the second strand (ss2) may be linked directly or indirectly. When the first strand (ss1) and the second strand (ss2) are linked, the sensor (I) can be referred to as a single stranded nucleic acid sensor, the first strand (ss1) can be referred to as a first region, and the second strand (ss2) can be referred to as a second region, for example. The direct linkage denotes, for example, the state in which the 3′ end of one of the regions and the 5′ end of the other of the regions are directly bound. The indirect linkage denotes, for example, the state in which the 3′ end of one of the regions and the 5′ end of the other of the regions are linked through an intervening linker region, specifically, the 3′ end of one of the regions and the 5′ end of the intervening linker region are directly bound and the 3′ end of the intervening linker region and the 5′ end of the other of the regions are directly bound. The intervening linker region may be, for example, a nucleic acid sequence or a non-nucleic acid sequence and is preferably the former. The length of the intervening linker region is not particularly limited and is, for example, 1 to 60-mer.

As to the order of the first region, the second region, and the intervening linker region, for example, the first region, the intervening linker region, and the second region may be linked in this order from the 5′ side or from the 3′ side and the former order is preferable.

In the sensor (I), for example, one end of the first strand (ss1) or the second strand (ss2) may be linked to the transistor.

In the sensor (I), for example, a linker region may be further added to one end or both ends of one of the first strand (ss1) and the second strand (ss2). Hereinafter, the linker region added to the end is also referred to as an additional linker region. The length of the additional linker region is not particularly limited and is, for example, 1 to 60-mer. In this case, in the sensor (I), for example, an end of one of the first strand (ss1) and the second strand (ss2) may be linked to the transistor through an additional linker region.

In the sensor (I), one of the first strand (ss1) and the second strand (ss2) may be disposed in the transistor and the other of the first strand (ss1) and the second strand (ss2) may be served as a reagent. In this case, the strand disposed in the transistor is preferably the second strand (ss2), and the strand serving as the reagent is preferably the first strand (ss1).

In the case where one of the first strand (ss1) and the second strand (ss2) is disposed in the transistor and the other of the first strand (ss1) and the second strand (ss2) is served as a reagent in the sensor (I), for example, based on the mechanism described below, it is presumed that, in the presence of the reagent, formation of the conformation in the conformation-forming region (D) is controlled to be ON or OFF depending on the presence or absence of a target, thereby decreasing the number of nucleotide residues that compose the sensor within the range of Debye length of the transistor, for example. Note that the present invention is described with reference to an example in which the second strand (ss2) is disposed in the transistor. The present invention, however, is not limited to this mechanism. In the sensor (I), the binding region (A) does not form a more stable structure for binding to a target in the absence of the target, and, in response to annealing between the stem-forming region (S_{A}) and the binding region (A), the binding region (A) is blocked from forming the more stable structure and keeps a structure of not binding to a target. In accordance with this, the conformation-forming region (D) is inhibited from forming the conformation (switch-OFF) and annealing between the stem-forming region (S_{D}) and the conformation-forming region (D) is allowed. Thus, in the absence of the target, the first strand (ss1) hybridizes to the second strand (ss2) in the sensor (I). On the other hand, in the presence of a target, upon contact of the target to the binding region (A), the binding region (A) changes into the stable structure and annealing between the stem-forming region (SA) and the binding region (A) is not allowed in the sensor (I). In accordance with this, annealing between the conformation-forming region (D) and the stem-forming region (S_{D}) is not allowed, thereby forming the conformation in the conformation-forming region (D) (switch-ON). Since the annealing between the binding region (A) and the stem-forming region (S_{A}) is not allowed and the annealing between the conformation-forming region (D) and the stem-forming region (S_{D}) is not allowed, the first strand (ss1) does not hybridize to the second strand (ss2) and the first strand (ss1) can move out of the range of Debye length of the transistor. Thus, according to the sensor (I), since the number of nucleotides within Debye length in the presence of the target (i.e., in the state where the conformation is formed) decreases as compared to in the absence of the target (i.e., in the state where formation of the conformation is inhibited), a target analysis such as a qualitative analysis or a quantitative analysis can be achieved. Note that, although the present invention is described with reference to an example in which the second strand (ss2) is disposed in the transistor, the first strand (ss1) may be disposed in the transistor.

2. Nucleic Acid Sensor (II)

The nucleic acid sensor (II) is, for example, a single stranded nucleic acid sensor including the conformation-forming region (D) and the binding region (A). In the absence of a target, the conformation-forming region (D) is inhibited from forming the conformation. In the presence of the target, upon contact of the target to the binding region (A), the conformation-forming region (D) forms the conformation. In the state where the conformation is formed, the number of nucleotide residues that compose the nucleic acid sensor within the range of Debye length of the transistor increases as compared to the state where formation of the conformation is inhibited.

As shown in FIG. 2, in the sensor (II), based on the mechanism described below, it is presumed that formation of the conformation in the conformation-forming region (D) is controlled to be ON or OFF depending on the presence or absence of a target, thereby increasing the number of nucleotide residues that compose the sensor within the range of Debye length of the transistor, for example. The present invention, however, is not limited to this mechanism. As shown in (A) of FIG. 2, in the sensor (II), in the absence of a target, the conformation-forming region (D) is inhibited from forming the conformation (switch-OFF) in a molecule. On the other hand, in the sensor (II), in the presence of a target, upon contact of the target to the binding region (A), the binding region (A) changes into a more stable structure for binding to a target. In accordance with this, the conformation is formed in the conformation-forming region (D) (switch-ON). As shown in (B) of FIG. 2, in response to the change of the structure of the binding region (A) into the more stable structure and the formation of the conformation in the conformation-forming region (D), the sensor (II) is shrank toward the transistor side, for example. Thus, according to the sensor (II), since the number of nucleotides within Debye length in the presence of the target (i.e., in the state where the conformation is formed) increases as compared to in the absence of the target (i.e., in the state where formation of the conformation is inhibited), a target analysis such as a qualitative analysis or a quantitative analysis can be achieved.

Specifically, the sensor (II) is, for example, at least one sensor selected from the group consisting of the following nucleic acid sensors (i) to (v). The sensor (II) may include, for example, one of or two or more kinds of the sensors.

2-1. Nucleic Acid Sensor (i)

The sensor (i) is, for example, as follows. That is, the sensor (i) is a single stranded nucleic acid sensor including the conformation-forming region (D), the blocking region (B), and the binding region (A) in this order. The blocking region (B) is complementary to a partial region (Dp) of the conformation-forming region (D). A terminal region (Ab) of the binding region (A) on the blocking region (B) side is complementary to an adjacent region (Df) adjacent to the partial region (Dp) in the conformation-forming region (D) and is also complementary to a terminal region (Af) of the binding region (A) on the side opposite to the blocking region (B) side.

In the sensor (i), the conformation-forming region (D) is, for example, the single stranded type.

In the sensor (i), based on the mechanism described below, it is presumed that formation of the conformation in the conformation-forming region (D) is controlled to be ON or OFF depending on the presence or absence of a target, thereby increasing the number of nucleotide residues that compose the sensor within the range of Debye length of the transistor, for example. In the sensor (i), since the partial region (Dp) of the conformation-forming region (D) is complementary to the blocking region (B) and the adjacent region (Df) of the conformation-forming region (D) is complementary to the terminal region (Ab) of the binding region (A), owing to these complementary relationships, stems can be formed. Thus, in the absence of the target, the partial region (Dp) of the conformation-forming region (D) and the blocking region (B) form a stem and the adjacent region (Df) of the conformation-forming region (D) and the terminal region (Ab) of the binding region (A) form a stem. In response to formation of the former stem, the conformation-forming region (D) is inhibited from forming the conformation (switch-OFF). In response to formation of the latter stem, the binding region (A) is blocked from forming a more stable structure for binding to a target and keeps a structure of not binding to a target. On the other hand, in the presence of a target, upon contact of the target to the binding region (A), the binding region (A) changes into the more stable structure. In accordance with this, formation of the stem in the binding region (A) is released and the target binds to the binding region (A) that has been changed into the more stable structure. In response to the structural change of the binding region (A) in accordance with the releases of the formation of a stem in the binding region (A), formation of the stem with the conformation-forming region (D) is released, and the conformation-forming region (D) is changed into a more stable structure. As a result, conformation is formed in the conformation-forming region (D) (switch-ON). In response to the change of the structure of the binding region (A) into the more stable structure and the formation of the conformation in the conformation-forming region (D), the sensor (i) is shrank toward the transistor side, for example. Thus, according to the sensor (i), since the number of nucleotides within Debye length in the presence of the target (i.e., in the state where the conformation is formed) increases as compared to in the absence of the target (i.e., in the state where formation of the conformation is inhibited), a target analysis such as a qualitative analysis or a quantitative analysis can be achieved.

The sensor (i) may further include a stabilization region (S). In this case, preferably, the conformation-forming region (D), the blocking region (B), the binding region (A), and the stabilization region (S) are linked in this order. Hereinafter, in the case where the embodiment in which the single stranded nucleic acid sensor including the stabilization region (S) is described as the sensor (i), the stabilization region (S) is optional and the single stranded nucleic acid sensor may not include the stabilization region (S).

The stabilization region (S) is, for example, a sequence for stabilizing the structure in binding of the binding region (A) and a target. Preferably, the stabilization region (S) is, for example, complementary to the whole or a part of the blocking region (B). Specifically, the stabilization region (S) is preferably complementary to a terminal region (Ba) of the blocking region (B) on the binding region (A) side. In this case, for example, when the stable structure of the binding region (A) is formed in the presence of a target, a stem is also formed between the stabilization region (S) that binds to the binding region (A) and the terminal region (Ba) of the blocking region (B) that binds to the binding region (A). Owing to such stems formed in the regions that bind to the binding region (A), the stable structure of the binding region (A) that binds to a target is further stabilized.

In the sensor (i), the order of the conformation-forming region (D), the blocking region (B), the binding region (A), and the stabilization region (S), which is optional, is not particularly limited. For example, these regions may be linked in this order from the 5′ side or from the 3′ side, and the former order is preferable.

In the sensor (i), the conformation-forming region (D), the blocking region (B), the binding region (A), and the stabilization region (S), which is optional, may be linked indirectly by disposing a spacer sequence between adjacent regions, for example. Preferably, these regions are linked directly without involving spacer sequences.

The conformation-forming region (D) includes, as described above, a sequence complementary to the blocking region (B) and a sequence complementary to a part of the binding region (A). Furthermore, the blocking region (B) is, as described above, complementary to a part of the conformation-forming region (D). In the case where the sensor includes the stabilization region (S), the blocking region (B) is also complementary to the stabilization region (S).

The sequence and the length of the blocking region (B) are not particularly limited, and can be determined appropriately according to the sequence, the length, and the like of the conformation-forming region (D), for example.

The length of the blocking region (B) is not particularly limited, and the lower limit thereof is, for example, 1-mer, 2-mer, or 3-mer, the upper limit thereof is, for example, 20-mer, 15-mer, or 10-mer, and the length is, for example, in the range from 1 to 20-mer, 2 to 15-mer, or 3 to 10-mer.

As to the length of the partial region (Dp) of the conformation-forming region (D), the lower limit is, for example, 1-mer, 2-mer, or 3-mer, the upper limit is, for example, 20-mer, 15-mer, or 10-mer, and the length is, for example, in the range from 1 to 20-mer, 2 to 15-mer, or 3 to 10-mer. The length of the blocking region (B) and the length of the partial region (Dp) of the conformation-forming region (D) are preferably the same, for example.

In the sensor (i), the position of the partial region (Dp) in the conformation-forming region (D), i.e., the annealing region of the blocking region (B) in the conformation-forming region (D) is not particularly limited. In the case where the conformation-forming region (D), the blocking region (B), the binding region (A), and the stabilization region (S), which is optional, are linked in this order, the partial region (Dp) can be defined by the following conditions, for example.

As to the length of a region (Db) that is adjacent to the partial region (Dp) in the conformation-forming region (D) and is located between the blocking region (B) side end of the partial region (Dp) and the conformation-forming region (D) side end of the blocking region (B), the lower limit is, for example, 3-mer, 4-mer, or 5-mer, the upper limit is, for example, 40-mer, 30-mer, or 20-mer, and the length is, for example, in the range from 3 to 40-mer, 4 to 30-mer, or 5 to 20-mer.

As to the length of a region (Df) that is adjacent to the partial region (Dp) in the conformation-forming region (D) and is located remote from the blocking region (B) side, the lower limit is, for example, 0-mer, 1-mer, or 2-mer, the upper limit is, for example, 40-mer, 30-mer, or 20-mer, and the length is, for example, in the range from 0 to 40-mer, 1 to 30-mer, or 2 to 20-mer.

The terminal region (Ab) of the binding region (A) on the blocking region (B) side is, as described above, complementary to the adjacent region (Df) of the conformation-forming region (D). The terminal region (Ab) of the binding region (A) may be complementary to the whole region of the adjacent region (Df) of the conformation-forming region (D) or may be complementary to a partial region of the adjacent region (Df). In the latter case, preferably, the terminal region (Ab) of the binding region (A) is complementary to the terminal region of the adjacent region (Df) of the conformation-forming region (D) on the partial region (Dp) side.

The length of the terminal region (Ab) of the binding region (A) that is complementary to the adjacent region (Df) of the conformation-forming region (D) is not particularly limited, and the lower limit thereof is, for example, 1-mer, the upper limit thereof is, for example, 20-mer, 8-mer, or 3-mer, and the length is, for example, in the range from 1 to 20-mer, 1 to 8-mer, or 1 to 3-mer.

The stabilization region (S) is, as described above, complementary to the whole or a part of the blocking region (B), for example. Specifically, the stabilization region (S) is preferably complementary to the terminal region (Ba) of the blocking region (B) on the binding region (A) side.

The length of the sequence of the stabilization region (S) is not particularly limited and can be determined appropriately according to the sequence and the length of each of the blocking region (B) and the binding region (A), and the like, for example. The lower limit of the sequence of the stabilization region (S) is, for example, 0-mer or 1-mer, the upper limit thereof is, for example, 10-mer, 5-mer, or 3-mer, and the length is, for example, in the range from 0 to 10-mer, 1 to 5-mer, or 1 to 3-mer. On the other hand, for example, the length of the blocking region (B) and the length of the stabilization region (S) are the same when the stabilization region (S) is complementary to the whole of the blocking region (B), and, for example, the length of a part of the blocking region (B) (e.g. the terminal region (Ba)) and the length of the stabilization region (S) are the same when the stabilization region (S) is complementary to a part of the blocking region (B).

The full-length of the sensor (i) is not particularly limited, and the lower limit thereof is, for example, 25-mer, 35-mer, or 40-mer, the upper limit thereof is, for example, 200-mer, 120-mer, or 80-mer, and the length is, for example, in the range from 25 to 200-mer, 35 to 120-mer, or 40 to 80-mer.

One end of the sensor (i) may be linked to the transistor, for example.

The additional linker region may be added to one end or both ends of the nucleic acid sensor (i), for example. The length of the additional linker region is not particularly limited, and reference can be made to the above description, for example. In this case, one end of the sensor (i) may be linked to the transistor through the additional linker region, for example.

2-2. Nucleic Acid Sensor (ii)

The sensor (ii) is, for example, as follows. That is, the sensor (ii) is a single stranded nucleic acid sensor including the conformation-forming region (D), a blocking region (B), the binding region (A), and a stabilization region (S) in this order. The blocking region (B) is complementary to a partial region (Dp) of the conformation-forming region (D). A terminal region (Ba) of the blocking region (B) on the binding region (A) side is complementary to the stabilization region (S).

In the sensor (ii), the conformation-forming region (D) is, for example, the single stranded type.

In the sensor (ii), preferably, the binding region (A) is a sequence that does not cause intramolecular annealing required for binding to a target by itself. In the sensor (ii), in the presence of a target, preferably, in response to annealing between the terminal region (Ba) of the blocking region (B) adjacent to the binding region (A) and the stabilization region (S), the binding region (A), the terminal region (Ba), and the stabilization region (S) as a whole form a stable structure for binding to the target.

In the sensor (ii), based on the mechanism described below, it is presumed that formation of the conformation in the conformation-forming region (D) is controlled to be ON or OFF depending on the presence or absence of a target, thereby increasing the number of nucleotide residues that compose the sensor within the range of Debye length of the transistor, for example. The present invention, however, is not limited to this mechanism. In the sensor (ii), since the partial region (Dp) of the conformation-forming region (D) is complementary to the blocking region (B), owing to this complementary relationship, a stem can be formed. Thus, in the absence of the target, the partial region (Dp) of the conformation-forming region (D) and the blocking region (B) form a stem. In response to formation of the stem, the conformation-forming region (D) is inhibited from forming the conformation (switch-OFF). Since the binding region (A) is a sequence that does not cause intramolecular annealing required for binding to a target by itself, the binding region (A) is blocked from forming a more stable structure for binding to a target and keeps a structure of not binding to a target. On the other hand, in the presence of a target, upon contact of the target to the binding region (A), the binding region (A) changes into the more stable structure. In accordance with this, formation of the stem between blocking region (B) and the partial region (Dp) of the conformation-forming region (D) is released and a stem is newly formed in response to annealing between the terminal region (Ba) of the blocking region (B) and the stabilization region (S). This stem serves as intramolecular annealing required for binding the binding region (A) to a target, and the stem and the binding region (A) as a whole form the stable structure, thereby binding the target to the binding region (A). Then, in response to release of the formation of the stem between the blocking region (B) and the conformation-forming region (D), a conformation is newly formed in the conformation-forming region (D) by intramolecular annealing (switch-ON). In response to the change of the structure of the binding region (A) into the more stable structure and the formation of the conformation in the conformation-forming region (D), the sensor (ii) is shrank toward the transistor side, for example. Thus, according to the sensor (ii), since the number of nucleotides within Debye length in the presence of the target (i.e., in the state where the conformation is formed) increases as compared to in the absence of the target (i.e., in the state where formation of the conformation is inhibited), a target analysis such as a qualitative analysis or a quantitative analysis can be achieved.

In the sensor (ii), the order of the conformation-forming region (D), the blocking region (B), the binding region (A), and the stabilization region (S), which is optional, is not particularly limited. For example, these regions may be linked in this order from the 5′ side or from the 3′ side, and the former order is preferable.

Regarding the sensor (ii), reference can be made to the description as to the sensor (i), unless otherwise noted. In the sensor (ii), the conformation-forming region (D), the blocking region (B), and the stabilization region (S) are the same as those described in the description as to the sensor (i), for example.

The blocking region (B) has, as described above, a sequence complementary to the conformation-forming region (D) and the stabilization region (S). Specifically, the blocking region (B) is complementary to the partial region (Dp) of the conformation-forming region (D) and the terminal region (Ba) of the blocking region (B) on the binding region (A) side is complementary to the stabilization region (S).

The length of the terminal region (Ba) of the blocking region (B) complementary to the stabilization region (S) is not particularly limited, and the lower limit thereof is, for example, 1-mer, the upper limit thereof is, for example, 15-mer, 10-mer, or 3-mer, and the length is, for example, in the range from 1 to 10-mer, 1 to 5-mer, or 1 to 3-mer.

The full-length of the sensor (ii) is not particularly limited, and the lower limit thereof is, for example, 25-mer, 35-mer, or 40-mer, the upper limit thereof is, for example, 200-mer, 120-mer, or 80-mer, and the length is, for example, in the range from 25 to 200-mer, 35 to 120-mer, or 40 to 80-mer.

One end of the sensor (ii) may be linked to the transistor, for example.

The additional linker region may be added to one end or both ends of the nucleic acid sensor (ii), for example. The length of the additional linker region is not particularly limited, and reference can be made to the above description, for example. In this case, one end of the sensor (ii) may be linked to the transistor through the additional linker region, for example.

2-3. Nucleic Acid Sensor (iii)

The sensor (iii) is, for example, as follows. That is, the sensor (iii) is a single stranded nucleic acid sensor including the conformation-forming region (D), a stem-forming region (S_{D}), the binding region (A), and a stem-forming region (S_{A}). The stem-forming region (S_{D}) includes a sequence complementary to the conformation-forming region (D). The stem-forming region (S_{A}) includes a sequence complementary to the binding region (A).

In the sensor (ii), the conformation-forming region (D) is, for example, the single stranded type.

In the sensor (iii), based on the mechanism described below, it is presumed that formation of the conformation in the conformation-forming region (D) is controlled to be ON or OFF depending on the presence or absence of a target, thereby increasing the number of nucleotide residues that compose the sensor within the range of Debye length of the transistor, for example. The present invention, however, is not limited to this mechanism. In the sensor (iii), in the absence of the target, in response to annealing between the conformation-forming region (D) and the stem-forming region (SD) in a molecule, the conformation-forming region (D) is inhibited from forming the conformation (switch-OFF). Furthermore, in response to annealing between the binding region (A) and the stem-forming region (SA) in a molecule, the binding region (A) is blocked from forming a more stable structure for binding to a target and keeps a structure of not binding to a target. On the other hand, in the presence of a target, upon contact of the target to the binding region (A), annealing between the binding region (A) and the stem-forming region (S_{A}) is released and the binding region (A) changes into the more stable structure. In accordance with this, the annealing between the conformation-forming region (D) and the stem-forming region (S_{D}) is released, and the conformation is formed in the conformation-forming region (D) (switch-ON). In response to the change of the structure of the binding region (A) into the more stable structure and the formation of the conformation in the conformation-forming region (D), the sensor (iii) is shrank toward the transistor side, for example. Thus, according to the sensor (iii), since the number of nucleotides within Debye length in the presence of the target (i.e., in the state where the conformation is formed) increases as compared to in the absence of the target (i.e., in the state where formation of the conformation is inhibited), a target analysis such as a qualitative analysis or a quantitative analysis can be achieved.

Preferably, the whole or a part of the stem-forming region (S_{D}) has a sequence complementary to a part of the conformation-forming region (D), for example. Furthermore, preferably, the whole or a part of the stem-forming region (S_{A}) has a sequence complementary to a part of the binding region (A), for example.

In the sensor (iii), the order of the regions can be any order as long as annealing between the conformation-forming region (D) and the stem-forming region (S_{D}) and annealing between the binding region (A) and the stem-forming region (S_{A}) are allowed in a molecule. Specific examples of the order are as follows.
(1) 5′-A-S_{D}-D-S_{A}-3′
(2) 5′-S_{A}-D-S_{D}-A-3′
(3) 5′-D-S_{A}-A-S_{D}-3′
(4) 5′-S_{D}-A-S_{A}-D-3′

In the embodiments (1) to (4), for example, ON-OFF of formation of a conformation is controlled as described below. In the absence of a target, the binding region (A) and the stem-forming region (S_{A}) form a stem and the conformation-forming region (D) and the stem-forming region (S_{D}) form a stem, thereby inhibiting the conformation-forming region (D) from forming a conformation. In the presence of the target, upon contact of a target to the binding region (A), formation of each stem is released and the conformation is formed in the conformation-forming region (D).

In the embodiments (1) and (3), preferably, the stem-forming region (S_{D}) is complementary to the 3′ side region of the conformation-forming region (D) and the stem-forming region (S_{A}) is complementary to the 3′ side region of the binding region (A). In the embodiments (2) and (4), preferably, the stem-forming region (S_{D}) is complementary to the 5′ side region of the conformation-forming region (D) and the stem-forming region (S_{A}) is complementary to the 5′ side region of the binding region (A).

In the sensor (iii), for example, the regions may be linked to each other directly or indirectly. The direct linkage denotes the state in which the 3′ end of one of the regions and the 5′ end of the other of the regions are linked directly, for example, and the indirect linkage denotes the state in which the 3′ end of one of the regions and the 5′ end of the other of the regions are linked indirectly through an intervening linker region, for example. The intervening linker region may be, for example, a nucleic acid sequence or a non-nucleic acid sequence and is preferably the former.

Preferably, the sensor (iii) includes two intervening linker regions noncomplementary to each other as the intervening linker regions, for example. The position of each of two intervening linker regions is not particularly limited.

Specific examples in which each of the embodiments (I) to (4) further includes two intervening linker regions are as follows. In the following examples, the intervening linker region that links to the binding region (A) is referred to as (L₁) and the intervening linker region that links to the conformation-forming region (D) is referred to as (L₂). The sensor (iii) may include both of the (L₁) and the (L₂) or either one of them as intervening linker region(s), for example.
(1′) 5′-A-L₁-S_{D}-D-L₂-S_{A}-3′
(2′) 5′-S_{A}-L₂-D-S_{D}-L₁-A-3′
(3′) 5′-D-L₂-S_{A}-A-L₁-S_{D}-3′
(4′) 5′-S_{D}-L₁-A-S_{A}-L₂-D-3′

In the embodiments (1′) to (4′), for example, ON-OFF of formation of a conformation is controlled as described below. In the absence of a target, for example, the binding region (A) and the stem-forming region (S_{A}) form a stem and the conformation-forming region (D) and the stem-forming region (S_{D}) form a stem, and the intervening linker region (L₁) and the intervening linker region (L₂) form an internal loop between these stems, thereby inhibiting the conformation-forming region (D) from forming a conformation. In the presence of the target, upon contact of a target to the binding region (A), formation of each stem is released and the conformation is formed in the conformation-forming region (D).

In the sensor (iii), the length of each of the stem-forming region (S_{A}) and the stem-forming region (S_{D}) is not particularly limited. The length of the stem-forming region (S_{A}) is, for example, 1 to 60-mer, 1 to 10-mer, or 1 to 7-mer. The length of the stem-forming region (S_{D}) is, for example, 1 to 30-mer, 0 to 10-mer, 1 to 10-mer, 0 to 7-mer, or 1 to 7-mer. The length of the stem-forming region (S_{A}) and the length of the stem-forming region (S_{D}) may be the same, the former may be longer than the latter, or the latter may be longer than the former.

The length of each of the intervening linker regions (L₁) and (L₂) is not particularly limited. The length of each of the intervening linker regions (L₁) and (L₂) is, for example, 0 to 30-mer, 1 to 30-mer, 1 to 15-mer, or 1 to 6-mer. The length of the intervening linker region (L₁) and the length of the intervening linker region (L₂) may be identical to or different from each other, for example. In the latter case, the difference between the length of the intervening linker region (L₁) and the length of the intervening linker region (L₂) is not particularly limited and is, for example, 1 to 10-mer, 1 or 2-mer, or 1-mer.

The length of the sensor (iii) is not particularly limited. The length of the sensor (iii) is, for example, 40 to 120-mer, 45 to 100-mer, or 50 to 80-mer.

One end of the sensor (iii) may be linked to the transistor, for example.

The additional linker region may be added to one end or both ends of the nucleic acid sensor (iii), for example. The length of the additional linker region is not particularly limited, and reference can be made to the above description, for example. In this case, one end of the sensor (iii) may be linked to the transistor through the additional linker region, for example.

2-4. Nucleic Acid Sensor (iv)

The sensor (iv) is, for example, as follows. That is, the sensor (iv) is a single stranded nucleic acid sensor including the conformation-forming region (D) and the binding region (A). The conformation-forming region (D) includes a first region (D1) and a second region (D2), and the first region (D1) and the second region (D2) form a conformation. The conformation-forming region (D) includes the first region (D1) on one end side of the binding region (A) and includes the second region (D2) on the other end side of the binding region (A).

In the sensor (iv), the conformation-forming region (D) is, for example, the double stranded type (hereinafter, also referred to as a “split type”). The split type conformation-forming region (D) is a molecule including the first region (D1) and the second region (D2), which form a conformation as a pair. In the sensor (iv), the sequences of the first region (D1) and the second region (D2) are not limited to particular sequences as long as they form the conformation and are preferably sequences that form a guanine quadruplex structure.

In the sensor (iv), based on the mechanism described below, it is presumed that formation of the conformation in the conformation-forming region (D) is controlled to be ON or OFF depending on the presence or absence of a target, thereby increasing the number of nucleotide residues that compose the sensor within the range of Debye length of the transistor, for example. The present invention, however, is not limited to this mechanism. In the sensor (iv), as described above, the first region (D1) and the second region (D2) that form a conformation as a pair are disposed remote from each other through the binding region (A). Since the first region (D1) and the second region (D2) are disposed at a distance, in the absence of the target, formation of a conformation between the first region (D1) and the second region (D2) is inhibited (switch-OFF). On the other hand, in the sensor (iv), in the presence of a target, upon contact of the target to the binding region (A), the structure of the binding region (A) changes into a more stable structure having a stem loop structure for binding to a target. In accordance with the structural change of the binding region (A), the first region (D1) and the second region (D2) approach to each other, and a conformation is formed between the first region (D1) and the second region (D2) (switch-ON). In response to the change of the structure of the binding region (A) into the more stable structure and the formation of the conformation in the conformation-forming region (D), the sensor (iv) is shrank toward the transistor side, for example. Thus, according to the sensor (iv), since the number of nucleotides within Debye length in the presence of the target (i.e., in the state where the conformation is formed) increases as compared to in the absence of the target (i.e., in the state where formation of the conformation is inhibited), a target analysis such as a qualitative analysis or a quantitative analysis can be achieved.

The sensor (iv), as described above, uses a double stranded type conformation-forming region (D), and the first region (D1) and the second region (D2) are disposed through the binding region (A). Thus, for example, there is no need to set conditions for each kind of aptamer and a desired aptamer can be set as the binding region (A). Accordingly, the sensor (iv) is superior in general versatility.

In the sensor (iv), it is only required that the first region (D1) and the second region (D2) are disposed through the binding region (A), and either of them may be disposed on the 5′ side or the 3′ side of the binding region (A). The present invention is described below with reference to an example in which the first region (D1) is disposed on the 5′ side of the binding region (A) and the second region (D2) is disposed on the 3′ side of the binding region (A) for the sake of convenience, unless otherwise stated.

In the sensor (iv), for example, the first region (D1) and the binding region (A) may be linked directly or indirectly and the second region (D2) and the binding region (A) may be linked directly or indirectly. The direct linkage denotes, for example, the state in which the 3′ end of one of the regions and the 5′ end of the other of the regions are directly bound. The indirect linkage denotes, for example, the state in which the 3′ end of one of the regions and the 5′ end of the other of the regions are linked through an intervening linker region, specifically, the 3′ end of one of the regions and the 5′ end of the intervening linker region are directly bound and the 3′ end of the intervening linker region and the 5′ end of the other of the regions are directly bound. The intervening linker region may be, for example, a nucleic acid sequence or a non-nucleic acid sequence and is preferably the former.

Preferably, the sensor (iv) includes, as described above, the intervening linker region (first linker region (L₁)) between the first region (D1) and the binding region (A) and the intervening linker region (second linker region (L₂)) between the second region (D2) and the binding region (A). The sensor (iv) may include one of the first linker region (L₁) and the second linker region (L₂) but preferably include both of them. In the case where the sensor (iv) includes both of the first linker region (L₁) and the second linker region (L₂), the lengths of them may be identical to or different from each other.

The length of the linker region is not particularly limited, and the lower limit thereof is, for example, 1-mer, 3-mer, 5-mer, 7-mer, or 9-mer and the upper limit thereof is, for example, 20-mer, 15-mer, or 10-mer.

Preferably, the base sequence of the first linker region (L₁) from the 5′ side and the base sequence of the second linker region (L₂) from the 3′ side are noncomplementary to each other, for example. In this case, it can be said that the base sequence of the first linker region (L₁) from the 5′ side and the base sequence of second linker region (L₂) from the 3′ side is a region that forms an internal loop in the molecule of the sensor (iv) in the state of being aligned. In this manner, owing to the first linker region (L₁) between the first region (D1) and the binding region (A) and the second linker region (L₂) between the second region (D2) and the binding region (A), which are noncomplementary to each other, for example, a sufficient distance can be kept between the first region (D1) and the second region (D2). Thus, for example, in the absence of the target, formation of conformation by the first region (D1) and the second region (D2) can be suppressed sufficiently, thereby sufficiently reducing the background based on formation of the conformation in the absence of a target.

The sensor (iv) can be, for example, represented by “D1-W-D2”. In the case where the sensor (iv) includes only the first linker region (L₁) as the intervening linker region, for example, “W” includes the first linker region (L₁) and the binding region (A) in this order from the 5′ side. In the case where the sensor (iv) includes only the second linker region (L₂), for example, “W” includes the binding region (A) and the second linker region (L₂) in this order from the 5′ side. In the case where the sensor (iv) includes both of the first linker region (L₁) and the second linker region (L₂), for example, “W” includes the first linker region (L₁), the binding region (A), and the second linker region (L₂) in this order from the 5′ side. In these cases, the sensor (iv) represented by D1-W-D2 can be represented, for example, by D1-L₁-A-D2, D1-A-L₂-D2, or D1 -L₁-A-L₂-D2.

Preferably, in the sensor (iv), the first region (D1) and the second region (D2) each have a sequence complementary to each other on the side opposite to the binding region (A), for example. Specifically, preferably, in the case where the first region (D1) is disposed on the 5′ side of the binding region (A), the first region (D1) and the second region (D2) each have a sequence complementary to each other at the 5′ end of the first region (D1) and the 3′ end of the second region (D2), for example. In the case where the first region (D1) is disposed on the 3′ side of the binding region (A), preferably, the first region (D1) and the second region (D2) each have a sequence complementary to each other at the 3′ end of the first region (D1) and the 5′ end of the second region (D2), for example. Owing to the complementary sequences at the end of each of the first region (D1) and the second region (D2), a stem structure can be formed between the sequences by intramolecular annealing. Thus, for example, in the presence of the target, in accordance with the structural change of the binding region (A) upon contact of the target; when the first region (D1) and the second region (D2) approach to each other, formation of the conformation by the first region (D1) and the second region (D2) becomes easier owing to the formation of the stem structure between the sequences.

The sensor (iv) can be, for example, represented by D1-W-D2 as described above. Specifically, the sensor (iv) can be represented by the following formula (I).

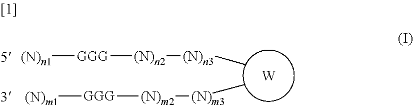

In the formula (I),

the sequence (N)ₙ₁-GGG-(N)ₙ₂-(N)ₙ₃- on the 5′ side represents the sequence (d1) of the first region (D1),

the sequence -(N)ₘ₃-(N)ₘ₂-GGG-(N)ₘ₁ on the 3′ side represents the sequence (d2) of the second region (D2),

W represents a region between the first region (D1) and the second region (D2) and includes the binding region (A), and

Ns each represent a base, and n1, n2, n3, m1, m2, and m3 each represent the number o repetitive bases N.

The formula (I) shows a state where the first region (D1) and the second region (D2) are intramolecularly aligned in the sensor (iv). This is a schematic view for showing the relationship between the sequence of the first region (D1) and the sequence of the second region (D2) and does not limit the present invention that the first region (D1) and the second region (D2) are in this state.

In the sequence (d1) of the first region (D1) and the sequence (d2) of the second region (D2), for example, (N)ₙ₁ and (N)ₘ₁ satisfy the following condition (1), (N)ₙ₂ and (N)ₘ₂ satisfy the following condition (2), and (N)ₙ₃ and (N)ₘ₃ satisfy the following condition (3).

Condition (1)

As to (N)ₙ₁ and (N)ₘ₁, the base sequence of (N)ₙ₁ from the 5′ side and the base sequence of (N)ₘ₁ from the 3′ side are complementary to each other, and n1 and m1 both are 0 or positive integers identical to each other.

Condition (2)

As to (N)ₙ₂ and (N)ₘ₂, the base sequence of (N)ₙ₂ from the 5′ side and the base sequence of (N)ₘ₂ from the 3′ side are noncomplementary to each other, and n2 and m2 each are a positive integer and may be identical to or different from each other.

Condition (3)

As to (N)ₙ₃ and (N)ₘ₃, n3 and m3 each are 3 or 4, may be identical to or different from each other, and include three bases G. In the case where n3 or m3 is 4 in (N)ₙ₃ and (N)ₘ₃, the second or the third base is a base H which is different from a base G.

The condition (1) is the condition of (N)ₙ₁ at the 5′ end and (N)ₘ₁ at the 3′ end in the case where the first region (D1) and the second region (D2) are aligned. In the condition (1), the base sequence of (N)ₙ₁ from the 5′ side and the base sequence (N)ₘ₁ from the 3′ side are complementary to each other, and the lengths thereof are the same. Since (N)ₙ₁ and (N)ₘ₁ are the complementary sequences of the same length, it can be said that they are stem regions that form a stem in the state of being aligned.

It is only required that n1 and m1 both are 0 or positive integers identical to each other. n1 and m1 each are, for example, 0 or 1 to 10 and each preferably are 1, 2, or 3.

The condition (2) is the condition of (N)ₙ₂ and (N)ₘ₂ in the case where the first region (D1) and the second region (D2) are aligned. In the condition (2), the base sequence of (N)ₙ₂ and the base sequence of (N)ₘ₂ are noncomplementary to each other, and the lengths thereof may be identical to or different from each other. Since (N)ₙ₂ and (N)ₘ₂ are noncomplementary sequences, it can be said that they are regions that form an internal loop in the state of being aligned.

n2 and m2 each are a positive integer, each are, for example, 1 to 10, and each are preferably 1 or 2. n2 and m2 may be identical to or different from each other. n2 and m2 satisfy, for example, any of the following conditions: n2=m2, n2>m2, and n2<m2. Preferably, n2 and m2 satisfy the following condition: n2>m2 or n2<m2.

The condition (3) is the condition of (N)ₙ₃ and (N)ₘ₃ in the case where the first region (D1) and the second region (D2) are aligned. In the condition (3), the base sequence of (N)ₙ₃ and the base sequence of (N)ₘ₃ each have a length of 3-mer or 4-mer including three bases G, and may be identical to or different from each other. In the case where n3 or m3 is 4 in (N)ₙ₃ and (N)ₘ₃, the second or the third base is a base H which is different from a base G. (N)ₙ₃ and (N)ₘ₃ each including three bases G are G-forming regions (G) that form a G-quartet structure together with GGG between (N)ₙ₁ and (N)ₙ₂ and GGG between (N)ₘ₁ and (N)ₘ₂.

n3 and m3 satisfy, for example, any of the following conditions: n3=m3, n3>m3, and n3<m3. Preferably, n3 and m3 satisfy the condition: n3>m3 or n3<m3.

The base H which is different from a base G can be, for example, A, C, T, or U and is preferably A, C, or T.

Specific examples of the condition (3) include the following conditions (3-1), (3-2), and (3-3).

Condition (3-1)

the sequence of one of (N)ₙ₃ and (N)ₘ₃ from the 5′ side is GHGG and the sequence of the other of (N)ₙ₃ and (N)ₘ₃ from the 5′ side is GGG.

Condition (3-2)

the sequence of one of (N)ₙ₃ and (N)ₘ₃ from the 5′ side is GGHG and the sequence of the other of (N)ₙ₃ and (N)ₘ₃ from the 5′ side is GGG.

Condition (3-3)

the sequence of each of (N)ₙ₃ and (N)ₘ₃ is GGG.

The length of the first region (D1) is not particularly limited, and the lower limit thereof is, for example, 7-mer, 8-mer, or 10-mer, the upper limit thereof is, for example, 30-mer, 20-mer, or 10-mer, and the length is, for example, in the range from 7 to 30-mer, 7 to 20-mer, or 7 to 10-mer. The length of the second region (D2) is not particularly limited, and the lower limit thereof is, for example, 7-mer, 8-mer, or 10-mer, the upper limit thereof is, for example, 30-mer, 20-mer, or 10-mer, and the length is, for example, in the range from 7 to 30-mer, 7 to 20-mer, or 7 to 10-mer. The length of the first region (D1) and the length of the second region (D2) may be identical to or different from each other.

The length of the sensor (iv) is not particularly limited, and the lower limit thereof is, for example, 25-mer, 30-mer, or 35-mer, the upper limit thereof is, for example, 200-mer, 100-mer, or 80-mer, and the length is, for example, in the range from 25 to 200-mer, 30 to 100-mer, or 35 to 80-mer.

One end of the sensor (iv) may be linked to the transistor, for example.

The additional linker region may be added to one end or both ends of the sensor (iv), for example. The length of the additional linker region is not particularly limited, and reference can be made to the above description, for example. In this case, one end of the sensor (iv) may be linked to the transistor through the additional linker region, for example.

2-5. Nucleic Acid Sensor (v)

The sensor (v) is, for example, as follows. That is, the sensor (v) is a single stranded nucleic acid sensor including the conformation-forming region (D) and the binding region (A) in this order. The conformation-forming region (D) and the binding region (A) each have a sequence complementary to each other.

In the sensor (v), the conformation-forming region (D) is, for example, the single stranded type.

In the sensor (v), based on the mechanism described below, it is presumed that formation of the conformation in the conformation-forming region (D) is controlled to be ON or OFF depending on the presence or absence of a target, thereby increasing the number of nucleotide residues that compose the sensor within the range of Debye length of the transistor, for example. The present invention, however, is not limited to this mechanism. In the sensor (v), in the absence of the target, in response to annealing between the conformation-forming region (D) and the binding region (A) in a molecule, the conformation-forming region (D) is inhibited from forming the conformation (switch-OFF). Furthermore, in response to annealing between the binding region (A) and the conformation-forming region (D) in a molecule, the binding region (A) is blocked from forming a more stable structure for binding to a target and keeps a structure of not binding to a target. On the other hand, in the sensor (v), in the presence of a target, upon contact of the target to the binding region (A), the structure of the binding region (A) changes into the more stable structure. In accordance with this, the annealing between the conformation-forming region (D) and the binding region (A) is released, and the conformation is formed in the conformation-forming region (D) (switch-ON). In response to the change of the structure of the binding region (A) into the more stable structure and the formation of the conformation in the conformation-forming region (D), the sensor (v) is shrank toward the transistor side, for example. Thus, according to the sensor (v), since the number of nucleotides within Debye length in the presence of the target (i.e., in the state where the conformation is formed) increases as compared to in the absence of the target (i.e., in the state where formation of the conformation is inhibited), a target analysis such as a qualitative analysis or a quantitative analysis can be achieved.

In the sensor (v), preferably, the sequence of the conformation-forming region (D) from the 5′ side and the sequence of the binding region (A) from the 3′ side each have a sequence complementary to each other. The complementary sequence in the conformation-forming region (D) and the complementary sequence in the binding region (A) each may be also referred to as a stem-forming region (S). The former complementary sequence in the conformation-forming region (D) can be also referred to as a stem-forming region (S_{A}) corresponding to the binding region (A), and the latter complementary sequence in the binding region (A) can be also referred to as a stem-forming region (S_{D}) corresponding to the conformation-forming region (D). Preferably, a part of the conformation-forming region (D) is the complementary sequence, i.e., the stem-forming region (S_{A}), for example, and a part of the binding region (A) is the complementary sequence, i.e., the stem-forming region (S_{D}), for example. The position of the complementary sequence in the conformation-forming region (D) and the position of the complementary sequence in the binding region (A) are not particularly limited.

In the sensor (v), the length of the complementary sequence in each of the conformation-forming region (D) and the binding region (A) is not particularly limited. The length of each of the complementary sequences is, for example, 1 to 30-mer, 1 to 10-mer, or 1 to 7-mer.

In the sensor (v), for example, the conformation-forming region (D) and the binding region (A) may be linked directly or indirectly. The direct linkage denotes the state in which the 3′ end of one of the regions and the 5′ end of the other of the regions are linked directly, for example, and the indirect linkage denotes the state in which the 3′ end of one of the regions and the 5′ end of the other of the regions are linked indirectly through a linker region, for example.

Hereinafter, the linker region that links the regions is also referred to as an intervening linker region. The intervening linker region may be, for example, a nucleic acid sequence or a non-nucleic acid sequence and is preferably the former. The length of the intervening linker region is not particularly limited, and is, for example, 0 to 20-mer, 1 to 10-mer, or 1 to 6-mer.

The length of the sensor (v) is not particularly limited. The length of the sensor (v) is, for example, 40 to 120-mer, 45 to 100-mer, or 50 to 80-mer.

One end of the sensor (v) may be linked to the transistor, for example.

The additional linker region may be added to one end or both ends of the sensor (v), for example. The length of the additional linker region is not particularly limited, and reference can be made to the above description, for example. In this case, one end of the sensor (v) may be linked to the transistor through the additional linker region, for example.

In the present invention, the sensor is a molecule containing nucleotide residues, and may be, for example, a molecule consisting only of nucleotide residues or a molecule containing nucleotide residues. Examples of the nucleotide include ribonucleotide, deoxyribonucleotide, and the derivatives thereof. Specifically, the sensor can be, for example, DNA containing deoxyribonucleotide and/or the derivative thereof, RNA containing ribonucleotide and/or the derivative thereof, or chimera (DNA/RNA) containing both of the former and the latter. Preferably, the sensor is DNA.

The nucleotide may contain either natural bases (inartificial bases) or unnatural bases (artificial bases) as bases, for example. Examples of the natural base include A, C, G, T, U, and the modified bases thereof. The modification can be, for example, methylation, fluorination, amination, or thiation. Examples of the unnatural base include 2′-fluoropyrimidine and 2′-O-methylpyrimidine, and specific examples thereof include 2′-fluorouracil, 2′-aminouracil, 2′-O-methyluracil, and 2′- thiouracil. The nucleotide may be, for example, a modified nucleotide, and examples of the modified nucleotide include 2′-methylated-uracil nucleotide residue, 2′-methylated-cytosine nucleotide residue, 2′-fluorinated-uracil nucleotide residue, 2′-fluorinated-cytosine nucleotide residue, 2′-aminated-uracil nucleotide residue, 2′-aminated-cytosine nucleotide residue, 2′-thiated-uracil nucleotide residue, and 2′-thiated-cytosine nucleotide residue. The sensor may contain non-nucleotides such as peptide nucleic acid (PNA) and locked nucleic acid (LNA), for example.

The sensor is disposed in the transistor. The sensor may be immobilized to the transistor directly or indirectly, for example. In the former case, preferably, the sensor is immobilized to the transistor at the end of the sensor, for example. In the latter case, for example, the sensor may be immobilized to the transistor though a linker for immobilization. The linker may be, for example, a nucleic acid sequence or a non-nucleic acid sequence, and can be the above-described additional linker region. In the case where the sensor is immobilized in the transistor, a site where the sensor is disposed can be referred to as a detection unit in the transistor.

The method for immobilization is not limited to particular methods and can be, for example, linkage by a chemical bond. As a specific example, by binding streptavidin or avidin to one of the transistor and the sensor and binding biotin to the other of the transistor and the sensor to utilize the bond between the former and the latter, the sensor is immobilized to the transistor.

Besides this, for example, a publicly known nucleic acid immobilization method can be adopted as the immobilization method. The method can be, for example, a method that utilizes photolithography, and reference can be made to the specification of U.S. Pat. No. 5,424,186 as a specific example. The method for immobilization can be, for example, a method of synthesizing the sensor on the transistor. This method can be, for example, a so-called spot method, and reference can be made to the specifications of U.S. Pat. No. 5,807,522 and JP H10(1998)-503841 A as specific examples.

In the present invention, the transistor is not limited to particular transistors. The transistor can be, for example, a transistor that can detect the change of the charge within the range of Debye length. As a specific example, the transistor can be a field effect transistor. Regarding the field effect transistor, for example, a publicly known field effect transistor can be used, and reference can be made to JP 2011-247795 A and WO 2014/024598 as specific examples.

In the present invention, the transistor includes, for example, a substrate, a source electrode, a drain electrode, and a detection unit, wherein the source electrode, the drain electrode, and the detection unit are disposed on the substrate, the detection unit is disposed between the source electrode and the drain electrode, and the nucleic acid sensor is disposed in the detection unit.

Regarding the substrate, the source electrode, and the drain electrode, reference can be made to the configuration of the publicly known field effect transistor. The transistor may include, for example, other components such as a gate electrode, a reference electrode, and an insulation film layer according to the kind of the field effect transistor. Regarding the other components, for example, reference can be made to the configuration of the publicly known field effect transistor.

The device of the present invention may be provided with a plurality of transistors, for example. In this case, preferably, each transistor is provided with the above-described detection unit, for example. In the sensor of the present invention, the number of sensors disposed in one detection unit is not particularly limited.

In the present invention, the Debye length denotes a distance within which the transistor can measure the charge. More specifically, the Debye length denotes a distance within which the detection unit of the transistor can measure the charge. The Debye length is not particularly limited and can be calculated by a common Debye length calculation expression. For example, the Debye length can be calculated by the following expression (1).

δ=(εε₀kT/2q2I)^{1/2} (1)

δ: Debye length

ε: relative permittivity

ε₀: permittivity in vacuum

k: Boltzmann constant

T: absolute temperature

q: charge

I: ionic strength

The use of the detection device of the present invention is not limited to particular uses, and the detection device of the present invention can be used for the target detection method of the present invention as described below.

<Target detection method>

The target detection method of the present invention is, as described above, characterized in that it includes the steps of: bringing a sample into contact with the detection device of the present invention; and detecting the increase or the decrease of the number of nucleotide residues that compose the nucleic acid sensor within the range of Debye length of the detection device to detect a target in the sample. The detection method of the present invention is characterized in that it uses the detection device of the present invention, and other configuration and conditions are not particularly limited. Regarding the detection method of the present invention, for example, reference can be made to the description as to the detection device of the present invention. In the detection method of the present invention, the detection can be the detection of the presence or absence of a target (for example, qualitative analysis) or the detection of the amount of a target (for example, quantitative analysis), and can be also referred to as, for example, an analysis method.

The sample is not limited to particular samples. The sample may be, for example, a sample that contains a target or a sample that may contain a target. Preferably, the sample is a liquid sample, for example. When a specimen is, for example, a liquid specimen, the specimen may be used as a sample as it is or a diluted solution obtained by mixing the specimen and a solvent may be used as a sample. When a specimen is, for example, a solid specimen, a powdery specimen, and the like, a mixture obtained by mixing the specimen and a solvent or a suspension obtained by suspending the specimen in a solvent may be used as a sample. The solvent is not limited to particular solvents, and examples thereof include water and buffer solutions. The specimen can be a specimen collected from a living body, a soil, seawater, river water, wastewater, food and beverage, purified water, air, or the like.

The contact step is a step of bringing a sample into contact with the detection device of the present invention. The contact can be conducted, for example, by bringing the sample into contact with the transistor of the detection device. Specifically, the contact can be conducted by bringing the sample into contact with the detection unit of the transistor. The contact conditions (temperature, time) in the contact step are not limited to particular conditions.

When the detection device contains the reagent, for example, the sample and the reagent may be separately brought into contact with the detection device or the mixture obtained by mixing the sample and reagent may be brought into contact with the device in the contact step. In the latter case, the detection method of the present invention includes a step of mixing the sample and the reagent to prepare a mixture and a step of bringing the mixture into contact with the detection device, for example. The mixing method is not limited to particular methods and can be a publicly known mixing method. For example, the mixing can be performed by bringing the reagent into contact with the sample. The mixing conditions (temperature, time) in the mixing step are not limited to particular conditions. The reagent can be, for example, the reagent containing the first strand (ss1) or the second strand (ss2).

In the detection step, by detecting the increase or the decrease of the number of nucleotide residues that compose the nucleic acid sensor within the range of Debye length of the detection device, a target in the sample is detected. In the presence of the target (i.e., in the state where the conformation is formed), the number of nucleotides within Debye length increases or decreases in the sensor as described above. The nucleotide residues that compose the sensor have, for example, a negative charge. Thus, in the presence of the target, the charge within the range of Debye length is increased or decreased as compared to in the absence of the target. Therefore, in the detection step, for example, by detecting the increase or decrease of the charge within the range of Debye length by using the detection device, the increase or decrease of the number of nucleotides within Debye length can be detected, i.e., a target in the sample can be detected. Hence, the detection step may include, for example, a step of measuring a charge within the range of Debye length of the detection device using the detection device and a step of detecting increase or decrease of the number of the nucleotide residues within the range of Debye length based on the charge (measured charge) and a reference charge to detect the target.

In the measuring step, the measurement of the charge can be, for example, the measurement of an electrical signal. The electrical signal can be measured, for example, by the transistor of the detection device. Examples of the electrical signal include a voltage and a current.

In the target detection step, the reference charge can be, for example, the charge within the range of Debye length in the absence of the target. By detecting the increase or decrease of the measured charge as compared to the reference charge, for example, the presence or absence of a target in the sample can be analyzed (qualitative analysis). By detecting the difference between the reference charge and the measured charge, for example, the amount of a target in the sample can be analyzed (quantitative analysis). Specifically, in the case where the number of nucleotide residues within the Debye length increases owing to the presence of a target, when the charge is significantly lower than the reference charge, it can be analyzed that the target is present, and when the charge is equivalent to or significantly higher than the reference charge, it can be analyzed that the target is not present. In the case where the number of the nucleotide residues within the Debye length decreases owing to the presence of a target, when the charge is significantly higher than the reference charge, it can be analyzed that the target is present, and when the charge is equivalent to or significantly lower than the reference charge, it can be analyzed that the target is not present.

In the target detection step, the reference charge can be a calibration curve that shows the correlation between the amount of the target and the measured charge. In this case, in the target detection step, for example, the amount of the target in the sample can be calculated on the basis of the measured charge.

The invention of the present application was described above with reference to the embodiments. However, the invention of the present application is not limited to the above-described embodiments. Various changes that can be understood by those skilled in the art can be made in the configurations and details of the invention of the present application within the scope of the invention of the present application.

This application claims priority from Japanese Patent Application No. 2015-214649 filed on Oct. 30, 2015. The entire subject matter of the Japanese Patent Application is incorporated herein by reference.

### INDUSTRIAL APPLICABILITY

According to the detection device of the present invention, for example, a target having no or almost no charge can be analyzed. Thus, the present invention is very useful in researches and tests in the various fields such as fields of clinical medical care, food, and environment, for example.

1. A detection device comprising:
a transistor provided with a nucleic acid sensor, wherein the nucleic acid sensor comprises:
a conformation-forming region (D) that forms a predetermined conformation; and
a binding region (A) that binds to a target,
in the absence of the target, the conformation-forming region (D) is inhibited from forming the conformation,
in the presence of the target, upon contact of the target to the binding region (A), the conformation-forming region (D) forms the conformation, and
in a state where the conformation is formed, the number of nucleotide residues that compose the nucleic acid sensor within a range of Debye length of the transistor increases or decreases as compared to a state where formation of the conformation is inhibited.

2. The detection device according to claim 1, wherein
the transistor comprises:
a substrate;
a source electrode;
a drain electrode; and
a detection unit,
the source electrode, the drain electrode, and the detection unit are disposed on the substrate,
the detection unit is disposed between the source electrode and the drain electrode, and
the nucleic acid sensor is disposed in the detection unit.

3. The detection device according to claim 1, wherein
the transistor is a transistor that can detect a change of a charge within the range of Debye length.

4. The detection device according to claim 1, wherein
the nucleic acid sensor is the following nucleic acid sensor (I):
(I) a double stranded nucleic acid sensor composed of a first strand (ss1) and a second strand (ss2), wherein
the first strand (ss1) comprises the conformation-forming region (D) and the binding region (A) in this order,
the second strand (ss2) comprises a stem-forming region (SD) and a stem-forming region (SA) in this order,
the stem-forming region (SD) has a sequence complementary to the conformation-forming region (D),
the stem-forming region (SA) has a sequence complementary to the binding region (A),
in the absence of the target, the conformation-forming region (D) is inhibited from forming the conformation and hybridizes to the second strand (ss2),
in the presence of the target, upon contact of the target to the binding region (A) of the first strand (ss1), the conformation-forming region (D) forms the conformation and the first strand (ss1) is dissociated from the second strand (ss2), and
in a state where the conformation is formed, the number of nucleotide residues that compose the nucleic acid sensor within the range of Debye length of the transistor decreases as compared to a state where formation of the conformation is inhibited.

5. The detection device according to claim 4, wherein
one of the first strand (ss1) and the second strand (ss2) of the nucleic acid sensor (I) is disposed in the transistor, and
the other of the first strand (ss1) and the second strand (ss2) is served as a reagent.

6. The detection device according to claim 1, wherein
the nucleic acid sensor is the following nucleic acid sensor (II):
(II) a single stranded nucleic acid sensor comprising the conformation-forming region (D) and the binding region (A), wherein
in the absence of the target, the conformation-forming region (D) is inhibited from forming the conformation,
in the presence of the target, upon contact of the target to the binding region (A), the conformation-forming region (D) forms the conformation, and
in a state where the conformation is formed, the number of nucleotide residues that compose the nucleic acid sensor within the range of Debye length of the transistor increases as compared to a state where formation of the conformation is inhibited.

7. The detection device according to claim 6, wherein
the nucleic acid sensor (II) is at least one nucleic acid sensor selected from the group consisting of the following nucleic acid sensors (i) to (v):
(i) a single stranded nucleic acid sensor comprising the conformation-forming region (D), a blocking region (B), and the binding region (A) in this order, wherein
the blocking region (B) is complementary to a partial region (Dp) of the conformation-forming region (D), and
a terminal region (Ab) of the binding region (A) on a blocking region (B) side is complementary to an adjacent region (Df) adjacent to the partial region (Dp) in the conformation-forming region (D) and is also complementary to a terminal region (Af) of the binding region (A) on a side opposite to the blocking region (B) side;
(ii) a single stranded nucleic acid sensor comprising the conformation-forming region (D), a blocking region (B), the binding region (A), and a stabilization region (S) in this order, wherein
the blocking region (B) is complementary to a partial region (Dp) of the conformation-forming region (D), and
a terminal region (Ba) of the blocking region (B) on a binding region (A) side is complementary to the stabilization region (S);
(iii) a single stranded nucleic acid sensor comprising the conformation-forming region (D), a stem-forming region (S_{D}), the binding region (A), and a stem-forming region (S_{A}), wherein
the stem-forming region (SD) has a sequence complementary to the conformation-forming region (D), and the stem-forming region (S_{A}) has a sequence complementary to the binding region (A);
(iv) a single stranded nucleic acid sensor comprising the conformation-forming region (D) and the binding region (A), wherein
the conformation-forming region (D) comprises a first region (D1) and a second region (D2), and the first region (D1) and the second region (D2) form a conformation, and
the conformation-forming region (D) comprises the first region (D1) on one end side of the binding region (A) and comprises the second region (D2) on the other end side of the binding region (A); and
(v) a single stranded nucleic acid sensor comprising the conformation-forming region (D) and the binding region (A) in this order, wherein
the conformation-forming region (D) and the binding region (A) each have a sequence complementary to each other.

8. The detection device according to claim 7, wherein
the single stranded nucleic acid sensor (i) or (ii) comprises the conformation-forming region (D), the blocking region (B), and the binding region (A) in this order from the 5′ side.

9. The detection device according to claim 7, wherein
the single stranded nucleic acid sensor (iii) comprises the stem-forming region (SD) and the stem-forming region (SA) as the stem-forming region (S),
the conformation-forming region (D) and the stem-forming region (SD) each have a sequence complementary to each other, and
the binding region (A) and the stem-forming region (SA) each have a sequence complementary to each other.

10. The detection device according to claim 7, wherein
in the single stranded nucleic acid sensor (iii), the conformation-forming region (D), the stem-forming region (S_{D}), the binding region (A), and the stem-forming region (S_{A}) are linked in the following order (1), (2), (3), or (4):
(1) order of the binding region (A), the stem-forming region (S_{D}), the conformation-forming region (D), and the stem-forming region (S_{A});
(2) order of the stem-forming region (S_{A}), the conformation-forming region (D), the stem-forming region (S_{D}), and the binding region (A);
(3) order of the conformation-forming region (D), the stem-forming region (S_{A}), the binding region (A), and the stem-forming region (S_{D}); and
(4) order of the stem-forming region (S_{D}), the binding region (A), the stem-forming region (S_{A}), and the conformation-forming region (D).

11. The detection device according to claim 7, wherein
in the single stranded nucleic acid sensor (iv), the first region (D1) and the second region (D2) each have a sequence complementary to each other on an end opposite to the binding region (A).

12. The detection device according to claim 7, wherein
in the single stranded nucleic acid sensor (v),
a sequence of the conformation-forming region (D) from a 5′ side and a sequence of the binding region (A) from a 3′ side each have a sequence complementary to each other.

13. The detection device according to claim 1, wherein
the conformation-forming region (D) is a G-forming region (G) that forms a G-quartet structure, and
the conformation is a G-quartet structure.

14. The detection device according to claim 1, wherein
the nucleic acid sensor comprises a linker region between the conformation-forming region (D) and the binding region (A).

15. The detection device according to claim 1, wherein
the nucleic acid sensor is linked to the transistor through a linker region.

16. A method for detecting a target, comprising the steps of:
bringing a sample into contact with the detection device according to claim 1; and
detecting increase or decrease of the number of nucleotide residues that compose the nucleic acid sensor within the range of Debye length of the detection device to detect a target in the sample.

17. The method according to claim 16, comprising the steps of:
mixing the sample and a reagent to prepare a mixture;
bringing the mixture into contact with the detection device; and
detecting increase or decrease of the number of nucleotide residues that compose the nucleic acid sensor within the range of Debye length of the detection device to detect a target in the sample, wherein
in the the detection device,
the nucleic acid sensor is the following nucleic acid sensor (I):
(I) a double stranded nucleic acid sensor composed of a first strand (ss1) and a second strand (ss2), wherein
the first strand (ss1) comprises the conformation-forming region (D) and the binding region (A) in this order,
the second strand (ss2) comprises a stem-forming region (S_{D}) and a stem-forming region (S) in this order,
the stem-forming region (S) has a sequence complementary to the conformation-forming region (D),
the stem-forming region (S_{A}) has a sequence complementary to the binding region (A),
in the absence of the target, the conformation-forming region (D) is inhibited from forming the conformation and hybridizes to the second strand (ss2),
in the presence of the target, upon contact of the target to the binding region (A) of the first strand (ss1), the conformation-forming region (D) forms the conformation and the first strand (ss1) is dissociated from the second strand (ss2), and
in a state where the conformation is formed, the number of nucleotide residues that compose the nucleic acid sensor within the range of Debye length of the transistor decreases as compared to a state where formation of the conformation is inhibited, wherein
one of the first strand (ss1) and the second strand (ss2) of the nucleic acid sensor (I) is disposed in the transistor, and
the other of the first strand (ss1) and the second strand (ss2) is served as the reagent.

18. The method according to claim 16, wherein
the detection step comprises the steps of:
measuring a charge within the range of Debye length of the detection device using the detection device; and
detecting increase or decrease of the number of the nucleotide residues within the range of Debye length based on the charge and a reference charge to detect the target.

19. The method according to claim 18, wherein
the charge is measured by measuring an electrical signal.

20. The method according to claim 19, wherein
the electrical signal is at least one of a voltage and a current.
